(19) United States
(12) Patent Application Publication
(10) Pub. No.: US 2006/0189914 A1
(43) Pub. Date: Aug. 24, 2006

(54) H-wrap elastic bandage

(76) Inventor: Jerome A. Slavitt, Pikesville, MD (US)

Correspondence Address: WHITEFORD, TAYLOR & PRESTON, LLP;ATTN: GREGORY M STONE, SEVEN SAINT PAUL STREET, BALTIMORE, MD, 21202-1626, US

(21) Appl. No.: 11/348,197

(22) Filed: Feb. 6, 2006

Related U.S. Application Data
(60) Provisional application No. 60/655,941, filed on Feb. 24, 2005.

(51) Int. Cl. A61L 15/00

(52) U.S. Cl. 602/75

(57) ABSTRACT

An elastic bandage is disclosed having narrow extension portion to allow for beginning compression at a specified pedal digit or finger. The elastic bandage can be wrapped around the identified digit and remain in place, as required, by integrated fastening hooks. The elastic bandage may be further wrapped around the foot or hand, as appropriate, to provide compression at the surgical site, as well as positional overcorrection, if required.

### CROSS REFERENCE TO RELATED APPLICATION

This application is based upon and claims benefit of co-pending U.S. Provisional Patent Application Serial No. 60/655,941 entitled “H-Wrap Elastic Bandage”, filed with the U.S. Patent and Trademark Office on Feb. 24, 2005 by the inventor herein, the specification of which is incorporated herein by reference.

### BACKGROUND OF THE INVENTION

1. Field of the Invention

This invention relates in general to a bandage device for providing specific compression to a pedal digit, such as the hallux or big toe, and the foot of a patient following surgical or other operations performed thereon. The bandage device can also be used for providing compression to the thumb or other digits of the hand and forearm.

2. Background of the Prior Art

In many instances following surgical treatment of a pedal digit, such as the hallux or big toe of a human being, for example, the correction of hallux abducto valgus or so-called bunions, it is customary and sometimes necessary to apply compression and reinforce stability at the surgical site to help reduce postoperative edema and mitigate against pain caused by pressure against the surgically treated toe, such as exerted, for example, by adjacent toes.

The mainstay for podiatric and orthopedic surgical dressing after the application of sterile gauze is the use of an elastic bandage, such as the well-known elastic bandage provided under the ACE® trademark. The elastic bandage is used to provide compression at the surgical site. Typically, the size of such elastic bandage is approximately 60-inches in length and 3-inches or 4-inches in width along its entire length.

In hallux valgus type surgical procedures, the applied compression generally begins about the distal circumference of the entire forefoot progressing proximally. Unfortunately, the appropriate starting point for compression, the hallux, has not been specifically addressed.

There remains a need, therefore, for an elastic bandage that can achieve the effect of applying compression, as well as positional overcorrection, if required, starting at the hallux or thumb, and progressing proximally to the rest of the foot, or hand.

Moreover, there remains a need for an elastic bandage that can attach to itself to enable the bandage to remain in place on the wearer.

### SUMMARY OF THE INVENTION

It is, therefore, an object of the present invention to provide an elastic bandage that avoids the disadvantages of the prior art.

It is another object of the present invention to provide an elastic bandage for providing compression at a surgical site on the hallux or thumb. A related object is to provide a compression device that targets the hallux or thumb as the primary point of compression. A further related object is to provide a device that can enable positional overcorrection, if warranted.

Another object of the present invention is to provide an elastic bandage that is economical to produce and uncomplicated in configuration. A related object of the present invention is to provide an elastic bandage that is simple to apply to the hallux or thumb according to the user's desires.

In accordance with the above objects, an elastic bandage is disclosed having a narrow extension portion to allow for beginning compression at a specified pedal digit or finger. The elastic bandage can be wrapped around the identified digit and remain in place, as required, by integrated fastening hooks. The elastic bandage may be further wrapped around the foot or hand, as appropriate, to provide compression at the surgical site and proximal components.

The various features of novelty that characterize the invention will be pointed out with particularity in the claims of this application.

### BRIEF DESCRIPTION OF THE DRAWINGS

The above and other features, aspects, and advantages of the present invention are considered in more detail, in relation to the following description of embodiments thereof shown in the accompanying drawings, in which:

FIG. 1 shows a plan view of an elastic bandage according to one embodiment of the present invention.

FIG. 2 shows a picture of a foot with post-surgical gauze wrap.

FIG. 3 shows a picture of an elastic bandage according to one embodiment of the present invention in the initial steps of application.

FIG. 4 shows a picture of an elastic bandage according to one embodiment of the present invention in the final steps of application.

### DETAILED DESCRIPTION OF THE INVENTION

The invention summarized above and defined by the enumerated claims may be better understood by referring to the following description, which should be read in conjunction with the accompanying drawings in which like reference numbers are used for like parts. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

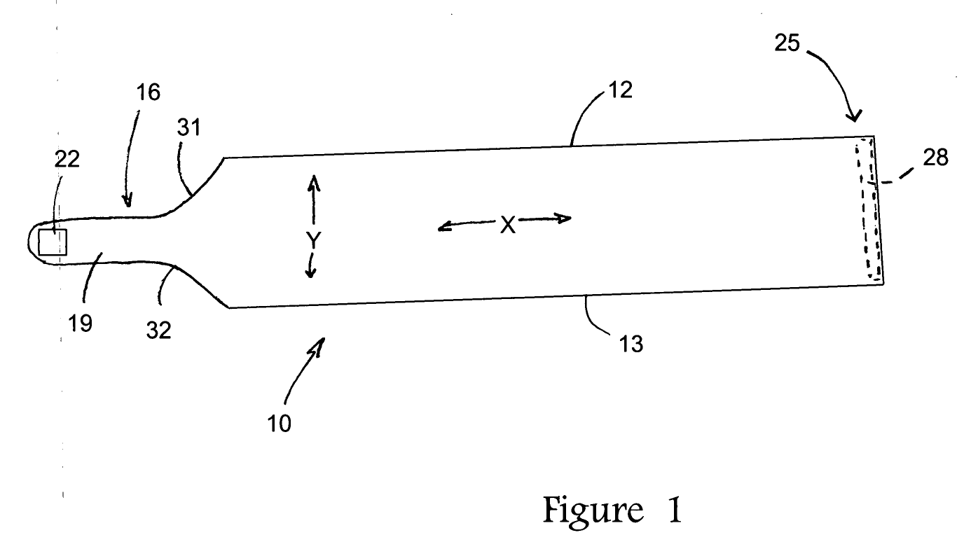

Referring to the drawings, FIG. 1 shows an elastic bandage, indicated generally as 10 according to an embodiment of the present invention. Elastic bandage 10 comprises an elongated flat body portion, made of soft, pliable material, such as cloth. The material should be of a weave that permits the bandage to stretch in the X direction, but not in the Y direction, as shown by the arrows in FIG. 1. In a preferred embodiment, the dimension of the overall length of elastic bandage 10 is significantly longer than the dimension of the width of said elastic bandage 10. For example, in some embodiments, elastic bandage 10 may be approximately 60 inches in length and only 3 or 4 inches in width. Other sizes can be used. The edges 12, 13 of elastic bandage 10 may be finished to avoid unraveling.

A first end 16 of elastic bandage 10 is tapered, having a narrow extension 19. such extension 19 should be substantially narrower than the remaining portion of bandage 10. For example, if the bandage 10 is approximately 60 inches in length and 3 or 4 inches in width, then the extension 19 may be approximately 8-inches long and 1 1/2 -inches wide. Other lengths and widths for extension 19 can be used. At the distal end of extension 19 is a first fastener 22, such as the hook portion of a hook and pile fastener, connected to one side of the elastic bandage 10. Such fastener 22 is designed to readily attach to the material of the elastic bandage 10.

A second end 25 of elastic bandage 10 has a second fastener 28 connected to the opposite side of the elastic bandage 10 as the first fastener 22. Second fastener 28, may also be designed to readily attach to the material of the elastic bandage 10. Other appropriate fasteners, known in the art, may be used.

In some embodiments, the edges 31, 32 of extension 19 may be finished to avoid unraveling. In other embodiments, the edges 31, 32 of extension 19 may have a binding covering such edges.

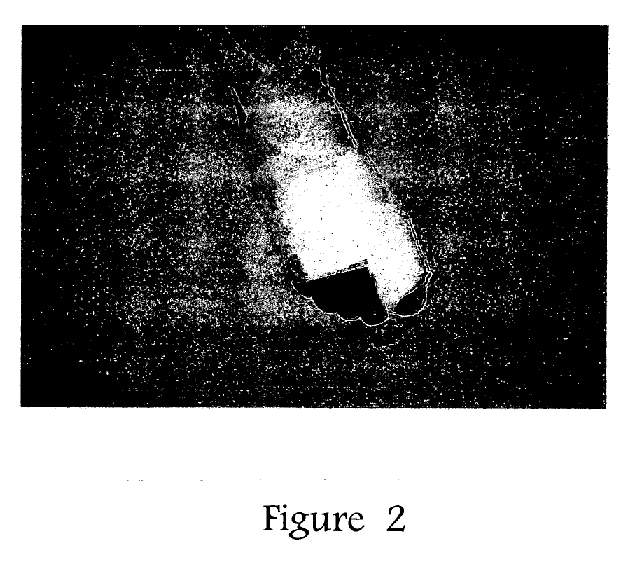

FIG. 2 shows a podiatric surgical dressing after the application of sterile gauze to the hallux of a right foot.

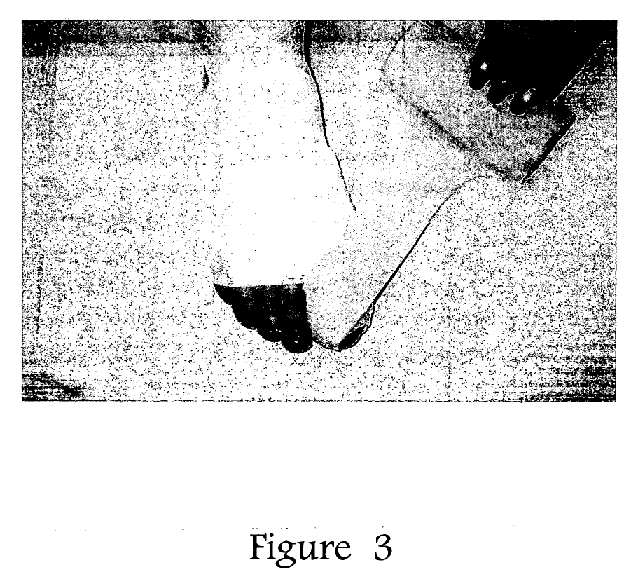

FIG. 3 shows the elastic bandage 10 being applied over the surgical dressing. Extension 19 is wrapped around the hallux and held in place by fastener 22 to allow for beginning compression at the hallux. In a preferred embodiment, the bandage 10 is placed such that the fastener faces away from the hallux. Due to the narrow configuration of extension 19, the elastic bandage 10 can provide over-correction if required during application of the bandage 10.

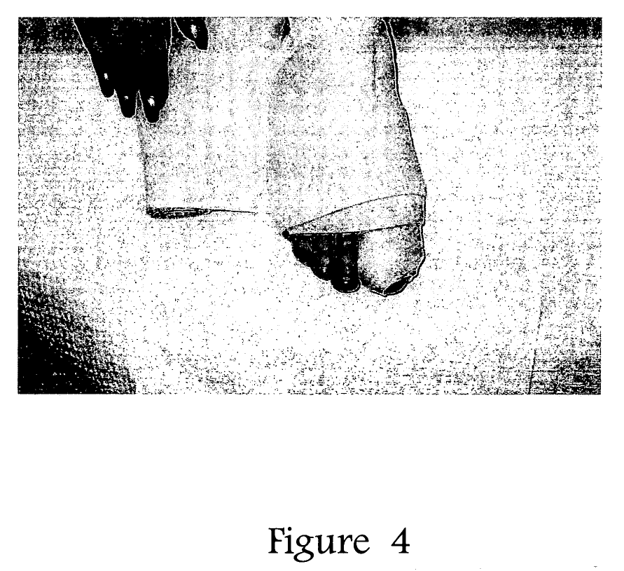

FIG. 4 shows further application of the elastic bandage 10. After initial application of elastic bandage 10 around the hallux, application of the bandage continues proximally up the foot and ankle as with any other elastic bandage. The bandage 10 is held in place by fastener 28.

While the invention has been described particularly for the hallux, the elastic bandage is equally applicable to any other pedal digit, as well as the thumb and digits of the hand.

It will be appreciated by persons skilled in the art that numerous variations and/or modifications may be made to the invention as shown in the specific embodiments without departing from the spirit or scope of the invention as broadly described. Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. It should be understood, therefore, that the invention may be practiced otherwise than as specifically set forth herein. The present embodiments are, therefore, to be considered in all respects as illustrative and not restrictive.

What is claimed is:

1. An elastic bandage, comprising:
an elongated flat body portion having a length dimension significantly longer than the width dimension of said body,
wherein one end of said body portion is tapered to a narrow extension.

2. The elastic bandage of claim 1, wherein said body is made of a material that enables said body to stretch in the direction of the length dimension but not in the direction of the width dimension.

3. The elastic bandage of claim 1, further comprising a first fastener connected to the distal end of said narrow extension.

4. The elastic bandage of claim 3, wherein said first fastener is designed to readily attach to the material of said body.

5. The elastic bandage of claim 3, further comprising a second fastener connected to the end of said body opposite said narrow extension.

6. The elastic bandage of claim 5, wherein said second fastener is designed to readily attach to the material of said body.

7. The elastic bandage of claim 5, wherein said second fastener is connected to said body of a side of said body opposite the side to which said first fastener is connected.

8. A method of compression wrapping a digit of a foot or hand, comprising the steps of:
providing a bandage having an elongated flat body portion with a length dimension significantly longer than the width dimension of said body, wherein one end of said body portion is tapered to a narrow extension;
wrapping said narrow extension around the digit of a foot or hand;
then continue wrapping the remainder of said bandage proximally along the foot or hand.

9. The method of claim 8, said bandage further comprising a first fastener connected to the distal end of said narrow extension, said method further comprising the steps of:
attaching said bandage to itself with said first fastener after a first wrapping of said narrow extension around said digit.

10. The method of claim 9, said bandage further comprising a second fastener connected to the end of said body opposite said narrow extension, said method further comprising the steps of:
attaching said bandage to itself with said second fastener after wrapping the remainder of said bandage proximally along the foot or hand.
